(19) United States
(12) Patent Application Publication
(10) Pub. No.: US 2004/0209301 A1
(43) Pub. Date: Oct. 21, 2004

(54) DNA chip having multi-layer film structure

(75) Inventors: Jae-ho You, Seoul (KR); Seung-ho Nam, Seongnam-si (KR); Hwan-young Choi, Yongin-si (KR); O-gweon Seo, Yongin-si (KR); Sang-hun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 10/825,297

(22) Filed: Apr. 16, 2004

(30) Foreign Application Priority Data
Apr. 16, 2003 (KR) 2003-23979

(51) Int. Cl. C12Q 1/68; C12M 1/34

(52) U.S. Cl. 435/6; 435/287.2

(57) ABSTRACT

Provided is a DNA chip having a multi-layer structure of thin films. The DNA chip comprises: a substrate; a high reflection region, having a higher refractive index than that of the substrate and including a thin film having a relatively low refractive index and a thin film having a relatively high refractive index sequentially stacked on a predetermined region of the substrate; a low reflection region, having a lower reflectance than that of the substrate and including a thin film having a relatively low refractive index stacked around the high reflection region on the substrate; a DNA probe fixed at least on the high reflection region. A hybridization reaction between the DNA probe and a target DNA labeled with a fluorescent dye occurs on the high reflection region.

### BACKGROUND OF THE INVENTION

This application claims the priority of Korean Patent Application No. 2003-23979, filed on Apr. 16, 2003, in the Korean Intellectual Property Office, the disclosure of which is incorporated herein in its entirety, by reference.

1. Field of the Invention

The present invention relates to a DNA chip, and more particularly, to a DNA chip having a multi-layer film structure that increases detection sensitivity of a hybridization signal generated from a hybridization reaction between a DNA probe and a target DNA.

2. Description of the Related Art

Biotechnology development has clarified DNA sequences that provide genetic information of organisms. Accordingly, the research and development of DNA chips for DNA sequence analysis and disease diagnosis have become an active R & D area.

A DNA chip enables a miniaturization of a DNA analysis system that allows one to perform a genetic analysis with a minute amount of sample and to examine many different DNA sequences on a target DNA simultaneously, thereby reducing analysis cost and rapidly providing genetic information. Also, the DNA chip is not only used to analyze vast amounts of genetic information simultaneously and in a short period of time, but also to examine relationships between genes.

Consequently, the applications of DNA chips are expected to contribute to the development of diagnostic tools for genetic diseases or cancer, mutation researches, virus detection, gene manifestation, and new medicines.

Moreover, the applications of the DNA chips in life related industries are expected to bring about revolutionary results. For example, a gene of a toxic material can be found using the DNA chip as a tool for detecting a microbe or an environmental contamination, thereby reducing the identification and manufacturing time of an antidote for a specific material.

In this way, the DNA chip can be applied to the production of antidotes against many toxic materials for medical and agricultural purposes, such as the production of low fat meat.

Referring to FIGS. 1 and 2, a conventional DNA chip 10 has a plurality of DNA probes 14 in a microarray arrangement on a substrate 11 formed of a silicon wafer or glass. More specifically, the DNA chip 10 is a fixed chip of DNA probes 14 in the form of spots 13 of several hundreds to several hundred thousands of predetermined locations on the substrate 11, each DNA probe 14 being a single-stranded DNA of a known DNA sequence. Generally, coating films 12 that include an amine or an aldehyde radical are formed for fixing the DNA probes 14 on a surface of the substrate 11. For analyzing a DNA, a target DNA 15 to be analyzed is reacted on the DNA chip 10. If the basic sequence of the target DNA 15 matches with the DNA probes 14, a double-stranded DNA is formed as a result of a hybridization reaction. At this time, the hybridization degrees may vary according to the complementary degree between the DNA probe 14 and the target DNA 15. Accordingly, the basic sequence of the target DNA 15 can be analyzed by detecting a hybridization degree at a certain spot 13 on the substrate 11. The hybridization degree can be detected by an optical method in which a signal generated from a fluorescent dye 16 is measured after the hybridization reaction between the target DNA 15, which is tagged by a fluorescent dye 16, and the DNA probe 14.

The DNA chip can be classified into an oligo chip and a cDNA chip according to the probe used, and also into a photolithography chip, a pin method spotting chip, and an ink jet method spotting chip according to the method of manufacturing. However, a common thing to all DNA chips is that a DNA probe with a single-stranded DNA of different kinds is fixed on the DNA chip and desired information is obtained by detecting a degree of a hybridization reaction between the target DNA and the DNA probe.

Therefore, the development of a DNA chip that can detect correctly a signal generated as a result of the hybridization reaction between the probe DNA 14 and the target DNA 15 is very important for obtaining a correct a genetic analysis.

In a conventional DNA chip, a signal emitted from a remained fluorescent dye 16 on a surface of a DNA chip after reacting between a DNA probe and the target DNA which is labeled with the fluorescent dye 16, is detected by using a confocal microscope or a CCD camera as disclosed in U.S. Pat. No. 6,141,096.

The confocal microscope provides high quality of image but slow signal detection, whereas the CCD camera provides a low quality of image but speedy signal detection. Accordingly, many investigations to increase the tagging amount of the fluorescent dye 16 for the target DNA are under way for providing speedy and accurate signal detection by an inexpensive scanner like the CCD type instead of a relatively expensive scanner like the confocal microscope. An example in this regard is the three dimensional hydrogel pad disclosed in U.S. Pat. No. 6,117,631.

However, the above optical detection methods have a drawback in that detection of a minute hybridization signal is difficult. Particularly, when background noise exists around the spot area, a correct detection of the hybridization signal is difficult.

Therefore, for a DNA chip that utilizes a complementary hybridization reaction between a DNA probe and a target DNA, there is a need to increase a detection sensitivity of a hybridization signal by making signal difference between the hybridization signal and the background signal as big as possible.

### SUMMARY OF THE INVENTION

To solve the above and other problems, the present invention provides a DNA chip having a multi-layer film structure in which a high reflection region and a low reflection region are formed to increase a detection sensitivity of a hybridization signal generated as a result of a hybridization reaction between a DNA probe and a target DNA.

According to an aspect of the present invention, the DNA chip comprises a substrate, a high reflection region having a higher reflectance than that of the substrate, the high reflection region comprising a thin film having a relatively low refractive index and a thin film having a relatively high refractive index sequentially stacked on a predetermined region of the substrate; a low reflection region having a lower reflectance than that of the substrate, the low reflection region comprising a thin film having a relatively low refractive index stacked around the high reflection region of the substrate; and a DNA probe fixed at least on the high reflection region on which a hybridization reaction between the DNA probe and a target DNA occurs.

Here, the high reflection region may be configured such that the low refractive index thin film and the high refractive index thin film are stacked alternately, and the low reflection region may be formed by multiple stacking of thin films of low refractive index.

Preferably, the thickness of the high refractive index thin film, in the high reflection region, is 70%˜130% of λ_{F}/4n_{H}, where λ_{F} is the emission wavelength of a fluorescent dye labeled to the target DNA, n_{H} and n_{L} are the refractive index of the high refractive index thin film and the refractive index of the low refractive index thin film, respectively, and the thickness of the low refractive index thin film in the low reflection region is 70%˜130% of λ_{F}/4n_{L}. Particularly, it is further preferable that the thickness of the high refractive index thin film is practically λ_{F}/4n_{H}, and that of the low refractive index thin film is practically λ_{F}/4n_{L}.

Also, preferably, the thickness of the low refractive index thin film is an odd multiple of λ_{F}/4n_{L} when λ_{F} is the emission wavelength of the fluorescent dye and n_{L} is the refractive index of the low refractive index thin film.

The high refractive index thin film may be formed of a metal oxide selected from the group consisting of TiO₂, ZrO₂, CeO₂ and Ta₂O₅ having a refractive index in the range of 2.0˜2.5, and the low refractive index thin film can be formed of silicon oxide.

The substrate may be formed of a material selected from the group consisting of silicon wafer, glass, quartz, and plastic.

A coating film for fixing the DNA probe may be formed on the surfaces of the high reflection region and the low reflection region, preferably, the coating film may be formed of one of an amine radical and an aldehyde radical.

### BRIEF DESCRIPTION OF THE DRAWINGS

The above aspects and advantages of the present invention will become more apparent by describing in detail an exemplary embodiment thereof with reference to the attached drawings in which:

FIG. 1 is a perspective view of a conventional DNA chip;

FIG. 2 is a cross-sectional view of a conventional DNA chip depicted in FIG. 1;

FIG. 3 is a perspective view of a DNA chip according to a first exemplary embodiment of the present invention;

FIG. 4 is a cross-sectional view of a multi-layer film structure of the DNA chip depicted in FIG. 3;

FIG. 5 is a cross-sectional view of a multi-layer film structure of a DNA chip according to a second exemplary embodiment of the present invention;

FIG. 6 is a cross-sectional view of a multi-layer film structure of a DNA chip according to a third exemplary embodiment of the present invention;

FIG. 7 is a graph showing a comparison of reflectance between the substrate and a high reflection region of a DNA chip depicted in FIG. 4 according to a first exemplary embodiment of the present invention; and

FIG. 8 is a graph showing a comparison of reflectance between the substrate and a low reflection region of a DNA chip depicted in FIG. 4 according to a first exemplary embodiment of the present invention.

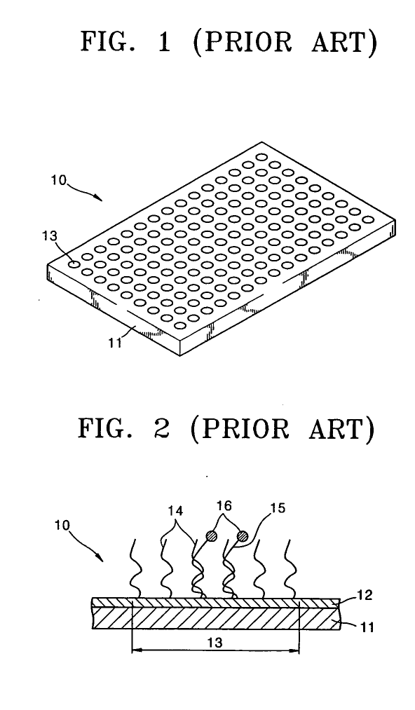

### DETAILED DESCRIPTION OF THE INVENTION

Hereinafter, a DNA chip having a multi-layer film structure according to embodiments of the present invention will be described more fully with reference to the accompanying drawings. To facilitate understanding, identical reference numerals have been used, where possible, to designate identical elements that are common to the figures.

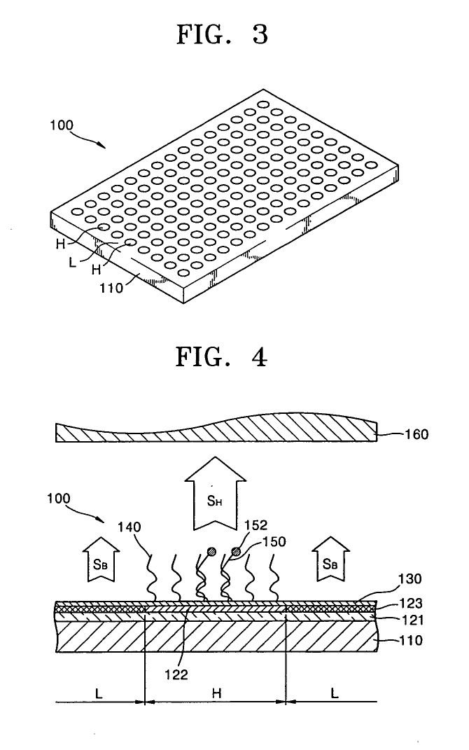

FIG. 3 is perspective view of a DNA chip according to a first exemplary embodiment of the present invention. FIG. 4 is a cross-sectional view of multi-layer film structure of the DNA chip depicted in FIG. 3.

Referring to FIGS. 3 and 4, the DNA chip 100 according to the first exemplary embodiment of the present invention comprises a substrate 110, a high reflection region H and a low reflection region L formed on the substrate 110, and a DNA probe 140 fixed at least on a surface of the high reflection region H.

The high reflection region H having a higher reflectance than that of the substrate 110 is formed on the substrate 110 in a microarray form. The low reflection region L having a lower reflectance than that of the substrate 110 is formed on the peripheral area of the high reflection region H on the substrate 110.

The substrate 110 can be formed of a silicon wafer with a refractive index of 3.5. Alternatively, a solid substrate such as glass, quartz, or plastic can be used for the substrate 110 instead of the silicon wafer.

A first thin film 121 having a relatively low refractive index is stacked on the substrate 110. The thin film can be formed of silicon oxide (SiO₂) having a refractive index of 1.45.

A second thin film 122 having a relatively high refractive index and a third thin film 123 having a relatively low refractive index are stacked on the first thin film 121. More specifically, the second thin film 122 having a high refractive index is formed on the first thin film 121 in the high reflection region H, and the third thin film 123 having a low refractive index is formed on the first thin film 121 in the low reflection region L. The second thin film 122 can be formed of titanium oxide (TiO₂) having a refractive index of 2.3, and the third thin film 123, as the first thin film 121, can be formed of silicon oxide (SiO₂) having a refractive index of 1.45. The second thin film 122 can also be formed of metal oxide such as not only TiO₂ but also ZrO₂, CeO₂, or Ta₂O₅ having a refractive index of 2.0˜2.5.

As described above, the high reflection region H has a configuration of a multi-layer film structure in which the first thin film 121 having a low refractive index and the second thin film 122 having a high refractive index are sequentially stacked. It is well known that the reflectance of the stacked films in the high reflection region H is higher than that of the substrate 110. On the other hand, the low reflection region L has a configuration of a multi-layer film structure in which the first and the third thin film 121 and 123 having a low refractive index are sequentially stacked.

Hereinafter, the reflectance of a multi-layer film structure will be described in brief referring to equations 1, 2, and 3.

Equation 1 is a matrix form of an amplitude B which is an electric vector, and an amplitude C which is a magnetic vector, of an incident light.

$$[ \begin{matrix}B \\ C\end{matrix} ] = \{ {\prod }_{r = 1}^{q} [ \begin{matrix}cos {δ}_{r} & i sin {δ}_{r} / {n}_{r} \\ i {n}_{r} sin {δ}_{r} & cos {δ}_{r}\end{matrix} ] \} [ \begin{matrix}1 \\ {n}_{m}\end{matrix} ] \qquad [ Equation 1 ]$$

where q represents the number of stacked thin films, nᵣ and nₘ represent the reflectance of the thin film and the substrate, respectively, and if a thickness of the thin film is d, then δᵣ is expressed as (2πnᵣ·d)/λ, where, λ is wavelength of the incident light.

Equation 2 for calculating the reflectance R of the thin film can be derived from equation 1.

$$R = ( \frac{{n}_{0} B - C}{{n}_{0 B} + C} ) {• ( \frac{{n}_{0} B - C}{{n}_{0} B + C} )}^{*} \qquad [ Equation 2 ]$$

If C/B is defined as admittance Y, equation 3 can be obtained from equation 2.

$$R = [ \frac{{n}_{0} - Y}{{n}_{0} + Y} ] • \overline{[ \frac{{n}_{0} - Y}{{n}_{0} + Y} ]} \qquad [ Equation 3 ]$$

From the equations 1, 2, and 3, it can be seen that the multi-layer film stacking the low refractive index thin film and high reflection thin film has higher reflectance than that of the substrate. Also, as the number of stacked thin films increases, the reflectance also gradually increases.

Also, it is well known that when an optical thickness of the low refractive index thin film and the high refractive index thin film respectively is equal to one quarter the wavelength of the incident light, the reflectance of the multi-layer film becomes the highest. Therefore, preferably, the low refractive index thin film and the high refractive index thin film respectively have an optical thickness satisfying the equation 4.

nᵣd=(¼)λ [Equation 4]

where nᵣ represents the refractive index of the thin film, d represents the thickness of the thin film, and λ represents the wavelength of the incident light.

Referring to FIG. 4, in the first exemplary embodiment of the present invention, the respective thickness of the first thin film 121 having low refractive index and the second thin film 122 having high refractive index in the high reflection region H can preferably be determined by using equation 4.

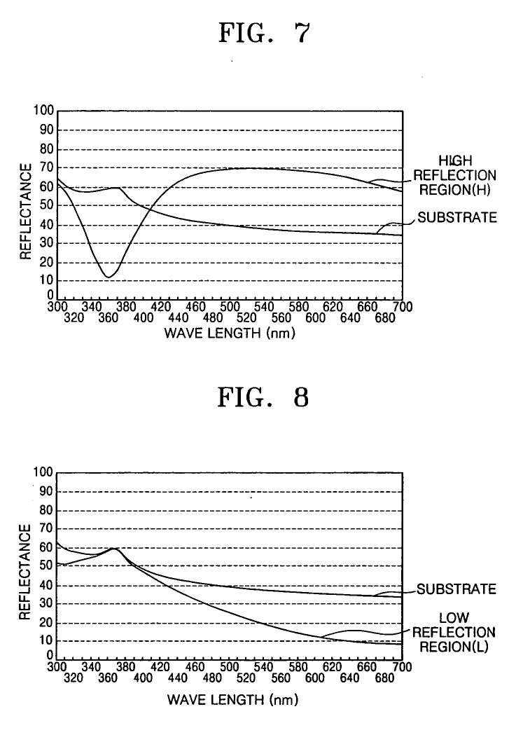

The thickness of the first thin film 121 can be defined approximately in a range of practically 70˜130 % of λ_{F}/4n_{L} where λ_{F} is an emission wavelength of a fluorescent dye 152 and n_{L} is a refractive index of the first thin film 121. The thickness of the second thin film 122 can be defined approximately in a range of practically 70˜130% of λ_{F}/4n_{H}, where n_{H} is a refractive index of the second thin film 122. It is preferable that the thickness of the first and the second thin film 121 and 122 are practically λ_{F}/4n_{L} and λ_{F}/4n_{H}, respectively, however, depositing the first and the second thin film 121 and 122 with the exact thicknesses of λ_{F}/4n_{L} and λ_{F}/4n_{H}, respectively, is very difficult in practice. However, when the thickness of the first and the second thin film 121 and 122 are in the range of practically 70˜130% of λ_{F}/4n_{L} and λ_{F}/4n_{H}, respectively, a reflectance higher than that of the substrate 110 can be obtained in the high reflection region H as shown in FIG. 7.

On the other hand, the low reflection region L does not have a multi-layer film characteristic like the high reflection region H because the low reflection region L is composed of the first and the third thin film 121 and 123 which have low refractive index. Therefore, the overall thickness of the low reflection region L is determined using the fact that the reflectance of the low reflection region L becomes equal to that of the substrate 110 when the optical thickness of a thin film is a multiple of one half the wavelength, i.e., even multiple of one fourth the wavelength, and the reflectance of the thin film becomes the lowest when the optical thickness of the thin film is an odd multiple of one fourth the wavelength.

Accordingly, the overall thickness of the low reflection region L is preferably an odd multiple of λ_{F}/4n_{L} where λ_{F} is the emission wavelength of a fluorescent dye, and the refractive index of the first and the third thin films 121 and 123 is n_{L}. However, when there is big step coverage between the overall thickness of the high reflection region H and the low reflection region L, the thickness of the low reflection region L can be adjusted to reduce the step coverage. In this case, the overall thickness of the low reflection region L can be adjusted to the reflectance of the low reflection region L is lower than the reflectance of the substrate 110.

According to the first exemplary embodiment of the present invention, the high reflection region H, has a higher reflectance than the substrate 110, however, the low reflection region L, has a lower reflectance than that of the substrate 110.

DNA probes 140 of a single strand and of known DNA sequence are fixed by a variety of methods on the high reflection region H. For this purpose, a coating film 130 composed of an amine or an aldehyde radical can be formed on the high reflection region H and the low reflection region L.

In FIG. 4, the DNA probes 140 are fixed only on the high reflection region H, but the DNA probes 140 can be fixed not only on the high reflection region H but also on the low reflection region L. In other words, in the former case, when a target DNA 150 is dispersed over the entire surface of the DNA chip 100, a hybridization reaction takes place only on the high reflection region H because the DNA probes 140 are fixed only on the high reflection region H. However, in the latter case, the target DNA 150 is dispersed only on the high reflection region H so that the hybridization reaction takes place only on the high reflection region H because the DNA probes 140 are fixed on both the high reflection region H and the low reflection region L.

When a target DNA 150 labeled with a fluorescent dye 152 reacts with DNA probes 140 on the surface of the DNA chip 100, according to the first exemplary embodiment of the present invention, a double-stranded DNA will be formed as a result of a hybridization reaction if the DNA sequence of the DNA probes 140 matches the DNA sequence of the target DNA 150. A hybridization degree depends on complementary degree between the DNA probe 140 and the target DNA 150. In a washing process, the target DNA 150 that formed a double strand with the DNA probe 140, by a hybridization reaction, remains on the DNA chip 100, and the target DNA 150 that did not form a double strand is removed. But an unwashed portion of the target DNA 150 labeled with the fluorescent dye 152 may remain in the low reflection region L.

Next, an exciting light from a light source (not shown) such as a light emitting diode (LED), a laser diode (LD), or a halogen lamp is irradiated to the DNA chip 100 to excite the fluorescent dye 152, and according to the Stock's law, a fluorescent signal having a longer wavelength than that of the exciting light, i.e., the emission wavelength λ_{F} is generated from the fluorescent dye 152, and the fluorescent signal is detected by a light detector 160.

Strength of the fluorescent signal SF can be expressed as equation 5.

$${S}_{F} = {\int }_{emission} F ( λ ) ⅆ λ \qquad [ Equation 5 ]$$

In equation 5, F(λ) is a function represents the amplitude of the fluorescent signal.

The hybridization signal from the high reflection region H detected by the light detector 160 includes not only the fluorescent signal generated from the fluorescent dye 152 but also a reflection signal due to the reflection of the fluorescent signal by the multi-layer films in the high reflection region H.

Therefore, the strength of the hybridization signal S_{H} can be expressed as in equation 6.

$${S}_{H} = {\int }_{emission} F ( λ ) ⅆ λ + {\int }_{reflection} F ( λ ) {R}_{H} ( λ ) ⅆ λ \qquad [ Equation 6 ]$$

In equation 6, R_{H}(λ) is a function which represents the reflectance R_{H} in the high reflection region H, and is derived from the equations 1, 2, and 3.

Referring to equation 6, the intensity of the reflection signal increases as the reflectance R_{H} increases in the high reflection region H, and accordingly, the intensity of the hybridization signal S_{H} also increases.

On the other hand, a background signal S_{B} of the low reflection region L detected by a light detector 160 includes the fluorescent signal generated from the resided fluorescent dye 152 and the reflection signal thereof, and a reflection signal of the exciting light irradiated from the light source toward the low reflection region L. Accordingly, the intensity of the background signal SB in the low reflection region can be expressed as equation 7.

$$\begin{matrix}{S}_{B} = {\int }_{emission} F ( λ ) ⅆ λ + {\int }_{reflection} F ( λ ) {R}_{L} ( λ ) ⅆ λ + \\ {\int }_{reflection} I ( λ ) {R}_{L} ( λ ) ⅆ λ\end{matrix} \qquad [ Equation 7 ]$$

In equation 7, I(λ) is a function which represents an amplitude of the exciting light irradiated from the light source, and R_{L}(λ) is a function which represents the reflectance R_{L} in the low reflection region L derived from equations 1, 2, and 3.

Referring to equation 7, as the reflectance R_{L} in the low reflection region L decreases, the intensity of the reflection signal decreases, and accordingly, the intensity of the background signal S_{B} also decreases.

As presented above, the intensity of the hybridization signal S_{H} as the result of the hybridization reaction between the DNA probe 140 and the target DNA 150 can further increase due to the high reflectance R_{H} in the high reflection region H, while the intensity of the background signal S_{B}, which acts as noise, can be further reduced due to the low reflectance R_{L} in the low reflection region L. Accordingly, the detection sensitivity for detecting the hybridization signal of the light detector 160 can be increased.

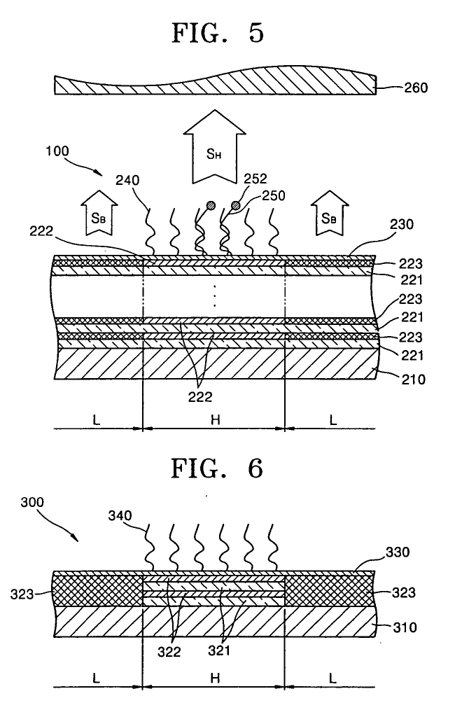

FIG. 5 is a cross-sectional view of a multi-layer structure of a DNA chip according to a second exemplary embodiment of the present invention. In the first exemplary embodiment of the present invention, the high reflection region and the low reflection region are composed of only two thin films. However, in the second exemplary embodiment depicted in FIG. 5, the high reflection region and the low reflection region are composed of a higher number of films.

Referring to FIG. 5, in the DNA chip 200 according to the second exemplary embodiment of the present invention, the high reflection region H is configured such that a first thin film 221 having a low refractive index and a second thin film 222 having a high refractive index are stacked alternately on a substrate 210. The low reflection region L is configured of the plural number of multi-layers of the first thin film 221 having a low refractive index and a third thin film 223 having a low refractive index alternately on the substrate 210.

The substrate 210 can be formed of a solid material such as a silicon wafer, glass, quartz, or plastic with a refractive index of 3.5 as in the first embodiment of the present invention. The second thin film 222 having a high refractive index may be formed of a TiO₂ and the first and the third thin film 221 and 223 having a low refractive index may be formed of a silicon oxide as described in the first exemplary embodiment of the present invention.

The thickness of the first thin film 221 having a low refractive index and the second thin film 222 having a high refractive index in the high reflection region H can be respectively determined as in the first exemplary embodiment. Also, the overall thickness of the low reflection region L can be determined as in the first embodiment. However, the low reflection region L has a thickness of odd multiple such as 3, 5, or 7 of λ_{F}/4n_{L} as the overall thickness of the high reflection region H increases.

A coating film 230 formed of an amine or an aldehyde radical can be formed for fixing the DNA probes 240 on surfaces of the high reflection region H and the low reflection region L. At least a single-stranded DNA probe 240 of a known DNA sequence is fixed on the high reflection region H.

In the DNA chip 200 configured according to the second exemplary embodiment of the present invention, since the high reflection region H has a alternately stacking structure of the first thin films 221 and the second thin films 222, the reflectance of the high reflection region H is high according to the equations 1, 2, and 3.

Accordingly, when a target DNA 250 labeled with a fluorescent dye 252 is reacted on the surface of the DNA chip 200, the intensity of the hybridization signal S_{H} generated from the hybridization reaction between the DNA probe 240 and the target DNA 250 increases, and the detection sensitivity of the hybridization signal detected by the light detector 260 also increases.

FIG. 6 is a cross-sectional view of a multi-layer structure of a DNA chip according to a third exemplary embodiment of the present invention. A main feature of the third exemplary embodiment of the present invention is that the low reflection region L is formed of a single layer of film unlike the low reflection regions L of the first and the second embodiment of the present invention.

Referring to FIG. 6, in the DNA chip 300 according to the third exemplary embodiment of the present invention, the high reflection region H comprises of a first thin film 321 having a low refractive index and a second thin film 322 having a high refractive index sequentially stacked on a substrate 310. The first thin films 321 and the second thin films 322 are stacked alternately as in the second exemplary embodiment.

The low reflection region L is composed of a single layer of a thin film 323 having a low refractive index disposed on the substrate 310.

The thicknesses and materials of the first and the second thin film 321 and 322 in the high reflection region H and the third thin film 323 in the low reflection region L of the third exemplary embodiment are the same as in the first and the second exemplary embodiments of the present invention. Also, the formation of the coating film 330 on the surface of the high reflection region H and the low reflection region L, and the fixation of the single-stranded DNA probe 340 of a known DNA sequence are the same as in the first and the second exemplary embodiment of the present invention.

Accordingly, the DNA chip 300 according to the third embodiment of the present invention also produces the same effect as in the previous exemplary embodiments. Moreover, the DNA chip 300 of the third embodiment can be easily manufactured since the low reflection region L is formed of a single layer of thin film 323 having a low refractive index.

Hereinafter, experimental results regarding the reflectance in the high reflection region H and the low reflection region L of the DNA chip 100 and the intensities of the hybridization signal and the background signal according to the reflectances, according to the first exemplary embodiment of the present invention as depicted in FIG. 4 will be described.

For this experiment, the substrate 110 was formed of a silicon wafer with a refractive index of 3.5, the first thin film 121 and the third thin film 123 were formed of a silicon oxide (SiO₂) with a refractive index n_{L} of 1.45, and the second thin film 122 was formed of a titanium oxide with a refractive index n_{H} of 2.3. The fluorescent dye 152 for the target DNA 150 has an emission wavelength λ_{F} of 550 nm.

Also, the thicknesses of the thin films were determined according to the method of determining the thickness in the high reflection region H as presented above. The first thin film 121 is stacked with a thickness of 94.18 nm, which is approximately 99% of λ_{F}/4n_{H}, and the second thin film 122 is stacked with a thickness of 57.65 nm, which is approximately 96% of λ_{F}/4n_{L}. On the other hand, it is preferable that no third thin film 123 is formed according to the method of determining the thickness in the low reflection region L, but the third thin film having a thickness of 29.26 nm was formed to reduce a step coverage between the high reflection region H and the low reflection region L as presented earlier in the description of the present invention. Therefore, the overall thickness of the low reflection region L was 123.44 nm, which is approximately 130% of λ_{F}/4n_{L}.

FIG. 7 shows the calculation results of the reflectance of the high reflection region H and the low reflection region L of the DNA chip 100 configured as presented above, according to the equations 1, 2, and 3.

Referring to FIG. 7, it is seen that the reflectance of the high reflection region H is higher than that of the substrate 110 in the wavelength range of 400˜700 nm. Particularly, the reflectance of the high reflection region H is the highest in the vicinity of a wavelength of 550 nm, which is the emission wavelength λ_{F} of the fluorescent dye 152.

Referring to FIG. 8, the reflectance of the low reflection region L is lower than that of the substrate 110 in the wavelength range of 400˜700 nm. The reflectance of the low reflection region L is the lowest in the vicinity of a wavelength of 700 nm. This is the result of forming the low reflection region L with a thickness of approximately 130% of λ_{F}/4n_{L}. However, the reflectance of the low reflection region L is lower than that of the substrate 110 in the vicinity of a wavelength of 550 nm, which is the emission wavelength λ_{F} of the fluorescent dye 152.

An intensity of the hybridization signal S_{H} in the high reflection region H having the above reflectance and an intensity of the background signal S_{B} in the low reflection region L having above refractive index were calculated according to equations 5, 6, and 7 and are summarized in Table 1.

TABLE 1

| | | Multi-layer | |
|---|---|---|---|
| | Conventional | of present | |
| Item | Si substrate | invention | Remark |
| Intensity of hybridization | 11282529 | 15395772 | 38.5% increase |
| signal (S_{H}) | | | |
| Intensity of Background | 9576788 | 7539443 | 21.3% decrease |
| signal (S_{B}) | | | |
| S_{H}/S_{B} | 1.17 | 2.05 | 74.2% increase |

Referring to Table 1, it is seen that the intensity of the hybridization signal S_{H} detected in the high reflection region H of the DNA chip according to the present invention has increased by approximately 38.5% and the intensity of the background signal S_{B} detected in the low reflection region L has decreased by approximately 21.3% compared with the case of using the conventional Si substrate.

Accordingly, the ratio S_{B}/R_{H} between the hybridization signal S_{H} and the background signal S_{B}, which is directly related to the detection sensitivity of the hybridization signal S_{H}, has increased by 74.2% in the DNA chip according to the present invention compared with the case of using the conventional Si substrate. The increased detection sensitivity of the DNA chip allows a correct detection of the hybridization signal, thereby enabling an efficient analysis of a DNA sequence of a target DNA.

As mentioned above, the DNA chip according to the present invention is formed of a multi-layer structure of thin films having a high reflection region and a low reflection region. As such, the hybridization signal generated from the hybridization reaction between the DNA probe and the target DNA has a higher intensity in the high reflection region due to the high reflectance of the high reflection region and the background signal in the low reflection region has a lower intensity due to the low reflectance of the low reflection region. Accordingly, a correct hybridization signal can be obtained by the increased detection sensitivity of the DNA chip.

While the present invention has been particularly shown and described with reference to exemplary embodiments thereof, it should not be construed as being limited to the embodiments set forth herein. Various modifications to the embodiments described can be made by those of skill in the art without departing from the scope of the present invention. Accordingly, the true scope of the present invention is determined not by the above description but by the appended claims and their equivalents.

What is claimed is:

1. A DNA chip comprising:
a substrate;
a high reflection region having a higher reflectance than that of the substrate, the high reflection region comprising a first film having a relatively low refractive index and a film having a relatively high refractive index stacked on a region of the substrate;
a low reflection region having a lower reflectance than that of the high reflection region, the low reflection region comprising a second film having a relatively low refractive index positioned around the high reflection region on the substrate; and
a DNA probe fixed on the high reflection region.

2. The DNA chip of claim 1, wherein the high reflection region is configured such that the first low refractive index film and the high refractive index film are stacked alternately on the substrate.

3. The DNA chip of claim 1, wherein the low reflection region is configured such that the second low refractive index film is stacked on the substrate.

4. The DNA chip of claim 1, wherein a thickness of the high refractive index film in the high reflection region is approximately 70%˜130% of λ_{F}/4n_{H}, where λ_{F} is an emission wavelength of a fluorescent dye labeled to a target DNA, n_{H} and n_{L} are refractive index of the high refractive index film and the refractive index of the first low refractive index film, respectively, and a thickness of the second low refractive index film in the low reflection region is approximately 70%˜130% of λ_{F}/4n_{L}.

5. The DNA chip of claim 4, wherein a thickness of the high refractive index film is λ_{F}/4n_{H}, and a thickness of the first low refractive index film is λ_{F}/4n_{L}.

6. The DNA chip of claim 1, wherein a thickness of the first low refractive index film is an odd multiple of λ_{F}/4n_{L}, where λ_{F} is an emission wavelength of a fluorescent dye labeled to the target DNA and n_{L} is a refractive index of the first low refractive index film.

7. The DNA chip of claim 1, wherein a high refractive index film is formed of a metal oxide selected from the group consisting of TiO₂, ZrO₂, CeO₂ and Ta₂O₅ and having a refractive index range of 2.0˜2.5.

8. The DNA chip of claim 1, wherein at least one of the first and second low refractive index films is formed of silicon oxide.

9. The DNA chip of claim 1, wherein the substrate is formed of a material selected from the group consisting of silicon wafer, glass, quartz and plastic.

10. The DNA chip of claim 1, further comprising a coating film formed on surfaces of the high reflection region and the low reflection region.

11. The DNA chip of claim 10, wherein the coating film is formed of one of an amine radical and an aldehyde radical.

12. The DNA chip of claim 1, further comprising a plurality of said high reflection regions arranged in a microarray.

13. The DNA chip of claim 1, wherein in said high reflection region said first film having a relatively low refractive index is positioned between said substrate and said film having a relatively high refractive index.

14. The DNA chip of claim 1, wherein said high reflection region comprises a plurality of said first films having a relatively low refractive index and a plurality of said films having a relatively high refractive index, and wherein said plurality of said high refractive index films and said first low refractive index films are stacked alternatively on said substrate.

15. The DNA chip of claim 1, wherein said low reflection region comprises a plurality of said second films.

16. The DNA chip of claim 1, wherein at least a portion of said second film in said low reflection region is part of said first film in said high reflection region.

17. The DNA chip of claim 1, wherein a thickness of said second film having a relatively low refractive index is the same as a total thickness of said film having a relatively high refractive index and said first film having a relatively low index of said high reflection region.

18. The DNA chip of claim 1, wherein the low reflection region has a lower reflectance than that of the substrate.

19. A DNA chip comprising:
a substrate;
a high reflection region having a higher reflectance than that of the substrate, wherein said high reflection region comprises a plurality of first films having a relatively low refractive index and a plurality of films having a relatively high refractive index alternatively stacked on a region of the substrate; and
a low reflection region having a lower reflectance than that of the high reflection region, wherein said low reflection region comprises a second film having a relatively low refractive index which surrounds the high reflection region on the substrate.

20. The DNA chip of claim 19, wherein the low reflection region has a lower reflectance than that of the substrate.

21. The DNA chip of claim 19, wherein a thickness of at least one of the high refractive index films in the high reflection region is approximately 70%˜130% of λ_{F}/4n_{H}, where λ_{F} is an emission wavelength of a fluorescent dye labeled to a target DNA, n_{H} and n_{L} are refractive index of the at least one high refractive index film and the refractive index at least one of the first low refractive index films, respectively, and a thickness of at least one second low refractive index films in the low reflection region is approximately 70%˜130% of λ_{F}/4n_{L}.

22. A DNA chip comprising:
a substrate;
a high reflection region having a higher reflectance than that of the substrate, wherein said high reflection region comprises at least one first film having a relatively low refractive index and at least one film having a relatively high refractive index positioned on a region of the substrate; and
a low reflection region having a lower reflectance than that of the high reflection region, wherein said low reflection region comprises a second film having a relatively low refractive index which surrounds the high reflection region on the substrate,
wherein a thickness of the high refractive index film in the high reflection region is approximately 70%˜130% of λ_{F}/4n_{H}, where λ_{F} is an emission wavelength of a fluorescent dye labeled to a target DNA, n_{H} and n_{L} are refractive index of the high refractive index film and the refractive index of the first low refractive index film, respectively, and a thickness of the second low refractive index film in the low reflection region is approximately 70%˜130% of λ_{F}/4n_{L}.

23. The DNA chip of claim 22, wherein the low reflection region has a lower reflectance than that of the substrate.
